(19) United States
(12) Patent Application Publication
(10) Pub. No.: US 2009/0163774 A1
(43) Pub. Date: Jun. 25, 2009

(54) Managment and Diagnostic System for Patient Monitoring and Symptom Analysis

(76) Inventors: Sudeesh Thatha, Bangalore (IN); M. R. Gowrishankar, Bangalore (IN); Susanta Patra, Bangalore (IN)

Correspondence Address: HONEYWELL INTERNATIONAL INC., 101 COLUMBIA ROAD, P O BOX 2245, MORRISTOWN, NJ, 07962-2245, US

(21) Appl. No.: 11/960,964

(22) Filed: Dec. 20, 2007

(51) Int. Cl. A61B 5/00; G06Q 50/00

(52) U.S. Cl. 600/301; 705/2

(57) ABSTRACT

A patient monitoring system collects data regarding physiological characteristics of an individual and analyzes that data to determine its validity and to identify any conditions present in the individual. The system can also perform a trend analysis to predict the onset of a condition in the individual. If a condition is present, an alert may be generated and the system may be used to diagnose the condition and determine a treatment plan based on a database of information collected from other patients using a similar system.

### FIELD OF THE INVENTION

This invention relates to systems that monitor the health and well-being of an individual. In particular, this invention relates to a patient monitoring system for assessing and predicting patient needs and generating proper alerts therefore.

### BACKGROUND OF THE INVENTION

With the new inventions in the field of Information Technology in the recent past, home-based disease-management programs are an important application of telemedicine. Home telecare and/or remote monitoring are rapidly evolving towards focused care in a home or community. Their primary role is providing support for the patient rather than the health professional. Typical applications include the management of chronic heart failure, Asthma, Diabetes and Hypertension.

In the current state of the art, Health Care Agencies install in-home monitoring systems and configure them to transmit various vital parameters at configured time intervals. After completing the necessary configurations, the monitoring systems transmit the different vital parameters to a Centralized Data Storage server. Once this data is available in the Central Station, assigned Nurses monitor these vital parameters continuously for the wellness of patients. Apart from monitoring these vitals or readings manually, nurses can even configure different limits (both high and low limits) for different vitals. The main advantage of these limits is, if any of the vitals' value crosses the configured limit boundaries then the system automatically generates an alert. With this auto generation of alerts, nurses can effectively monitor those patients whose vitals are abnormal.

However, for effective management and monitoring of health conscious people, chronically ill patients and/or elderly patients, it is essential to monitor various vital parameters in combination with each other. In addition to monitoring vital parameters individually and taking the necessary corrective actions, predicting trends in patient vitals can be very useful so that preventative measures can be taken prior to a patient becoming critical. By performing predictive analysis on the patient's historic data and generating trends therefrom, physicians would be able to take the appropriate preventative rather than reactionary steps. Therefore, a monitoring system that could assess trends in patient vitals and generate alerts based on those trends according to physician inputted conditions would be advantageous.

### BRIEF DESCRIPTION OF THE DRAWINGS

FIG. 1 is a block diagram of the logical architecture for an illustrative embodiment of the claimed invention.

FIG. 2 is a flowchart showing the architecture of the rule-based analysis of an embodiment of the claimed invention.

FIG. 3 is graphical representation of data collected by a weight sensor of an embodiment of the claimed invention over a period of time.

FIG. 4 is a graphical representation of data collected by a heart rate sensor of an embodiment of the claimed invention over a period of time.

FIG. 5 is a graphical representation of data collected by FVC, FEV1 and weight sensors of an embodiment of the claimed invention over a period of time.

### DETAILED DESCRIPTION OF THE ILLUSTRATIVE EMBODIMENT

While the present invention is susceptible of embodiment in various forms, there is shown in the drawings a presently preferred embodiment that is discussed in greater detail hereafter. It should be understood that the present disclosure is to be considered as an exemplification of the present invention, and is not intended to limit the invention to the specific embodiment illustrated. It should be further understood that the title of this section of this application (“Detailed Description of the Illustrative Embodiment”) relates to a requirement of the United States Patent Office, and should not be found to limit the subject matter disclosed herein.

The present embodiment of the claimed invention is directed to a patient monitoring system 10 composed of various physiological condition sensors 14 that wirelessly transmit physiological data regarding the patient 12 to a remotely located data storage server 20. Additionally, embodiments of the present invention can include a patient interface where healthcare professionals 18 can ask the patient 12 various questions about his or her condition and the answers are transmitted to the data storage server 20 as well. The data storage server 20 also contains historical data about the patient 12, physiological data regarding a plurality of other patients also being monitored by the system 10 and medication and treatment plan information for all patients.

After storage on the server 20, the data can then be analyzed in a variety of ways. A processor and first software analyze the data for validity and for the presence of any trends. Second software takes the results of the first software and at least some portion of the data and analyzes them with at least one logical rule to determine and validate whether a particular condition is present in the patient. The logical rule is created and stored by a healthcare professional using a conditional logic generator 40. The output of the second software then can be used to generate various types of alerts 44 to both the patient 12 and relevant healthcare professionals 18. A history of these generated alerts 44 is stored on the data storage server 20 and can be used in subsequent analyses. Finally, third software can be employed to do further analysis of the data stored on the data storage server 20 in order to classify patients by a particular condition, criticality, and/or treatment plan so that patients with similar conditions and/or treatment plans can be readily identified. This will help the healthcare professional to compare patient progress on a particular treatment plan and eventually assess the best medication or treatment plan for a particular disease.

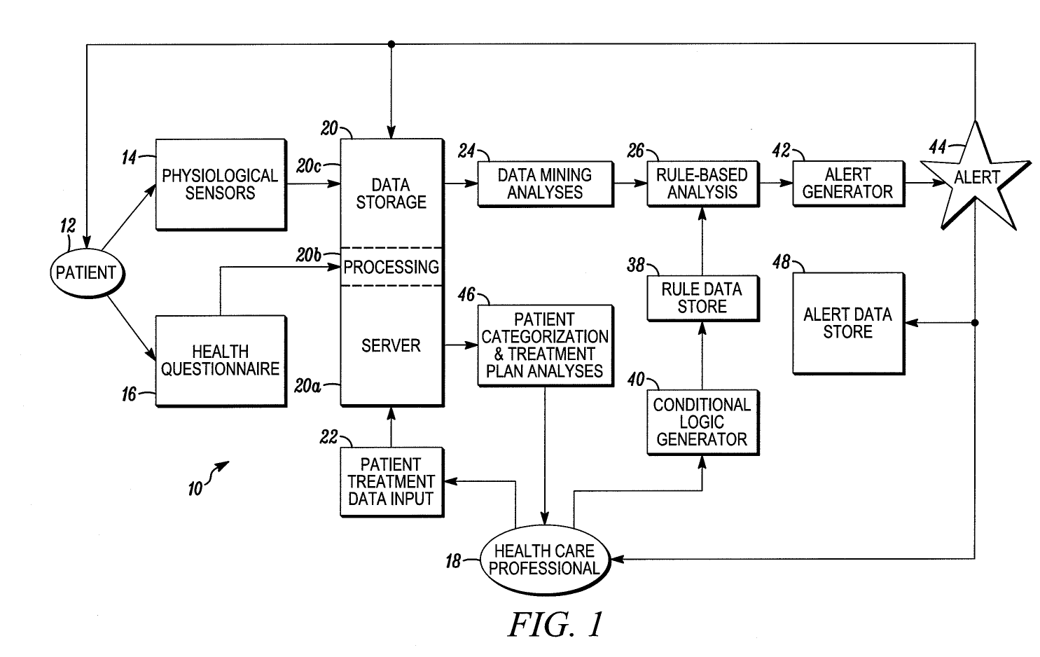

Referring now to FIG. 1, a block diagram depicting the architecture of the patient monitoring system 10 is shown. First, data regarding a patient's 12 physiological characteristics is acquired through a plurality of physiological sensors 14. These physiological sensors 14 can include but are not limited to blood pressure sensors, heart rate sensors, lung function sensors, glucose sensors and PT/INR sensors. Additional data regarding the patient's 12 condition can be obtained through use of the health questionnaire 16. The health questionnaire 16 is comprised of an interactive patient interface as understood by those of ordinary skill in the art where the patient 12 can input answers to a series of questions pre-stored by the healthcare professional 18 or asked in real time via the interface or other means of communication. Once the data regarding the patient 12 has been collected, it is then transmitted wirelessly to a remotely located data storage module 20. The data storage module includes a server 20a where data can be stored, executable software 20b for processing data and a storage unit 20c which stores at least one database.

Once received by the storage module 20, the patient's data is processed into a database so it can be readily retrieved for further analysis. The storage unit 20c also stores previously collected data from the patient 12, physiological data collected from a plurality of other patients using similar systems and medication and treatment plan data for all patients. The medication and treatment plan data for a particular patient is input into the storage unit 20c by a healthcare professional 18 through a data input interface 22.

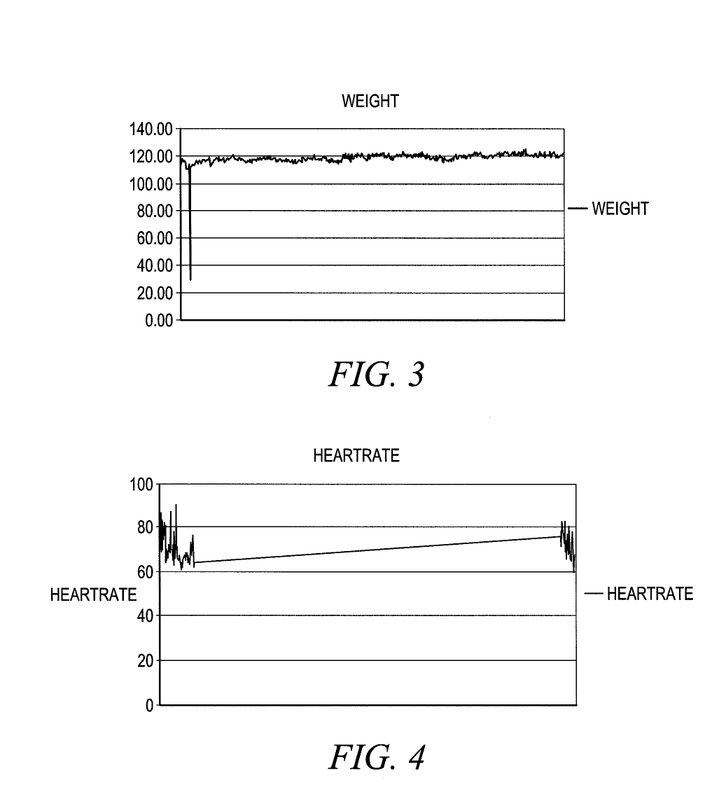

After processing at the storage module 20, the data undergoes data mining analyses 24 by a processor and first software. Due to various reasons including sensor malfunction, improper configuration or improper usage, there is a chance that data received by the storage module 20 is not valid. Therefore, the data is analyzed by the first software for validity and identification of false alerts using data mining and/or OLAP principles as understood by those of ordinary skill in the art. For instance, consider the scenario where the reading for a patient's weight suddenly drops from 116 pounds to 29 pounds as shown in FIG. 3. In the current stat of the art, an alert would be generated based on this occurrence. However, the first software is able to discard the data point of 29 pounds as invalid thus preventing a false alert from being generated.

Next, the first software uses curve fitting methodologies such as linear regression or non-linear regression and/or data mining principles to analyze for the presence of any trends in the data. Any identifiable trends or notable changes in the patient's 12 physiological characteristics are outputted by the first software. This way, rather than being able to alert the patient 12 or healthcare professional 18 only after the patient's 12 condition is severe, the system 10 can predict the onset of a severe condition and take preventative actions. For example, if the alert limit for heart rate was set at 80 and the received heart rate value is 79.5, no alert would be generated in the current state of the art. Moreover, the patient's heart rate could be rapidly increasing, as shown in FIG. 4, indicating the onset of a serious condition but no alert would be created. The first software mitigates these issues by its predictive analysis thus allowing proactive rather than reactive measures to be taken.

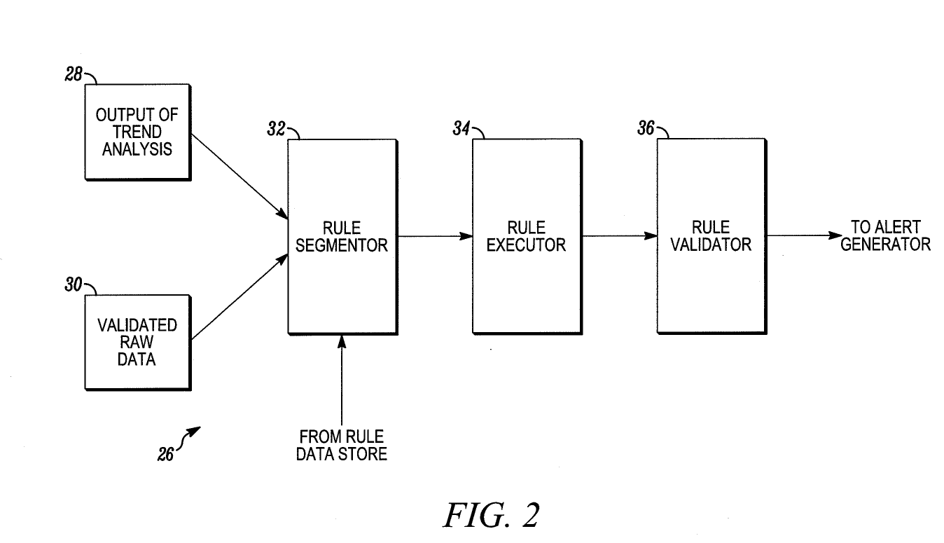

Next, the data and any output from the first software are input into rule-based analysis 26 performed by a second software. The detailed architecture of rule-based analysis 26 is shown in FIG. 2. The output of trend analysis 28 and validated raw data 30 from the first software are input into the rule segmentor 32. The rule segmentor 32 assesses what type of data has been input and retrieves the appropriate logical equations or rules from the rule data store 38. The rule executor 34 then parses and evaluates the data and/or data trends according to the conditions of the rule or rules. Finally, the rule validator 36 checks the outcome of the rule analysis to ensure it is consistent with other data and analyses for the patient 12. After this analysis, the second software then generates an output based on the results of the analysis which is then transmitted to the alert generator 42.

Prior to rule-based analysis 26, the healthcare professional 18 must create at least one rule to be analyzed by using the conditional logic generator 40. The conditional logic generator 40 is an interface component where the healthcare professional 18 can input one or more physiological parameters that will define a condition of interest in the patient 12. As those of ordinary skill in the art will understand, the conditional logic generator 40 will then generate logical rules based on the healthcare professional's 18 inputs and send them to the rule data store 38 for later use in rule-based analysis 26.

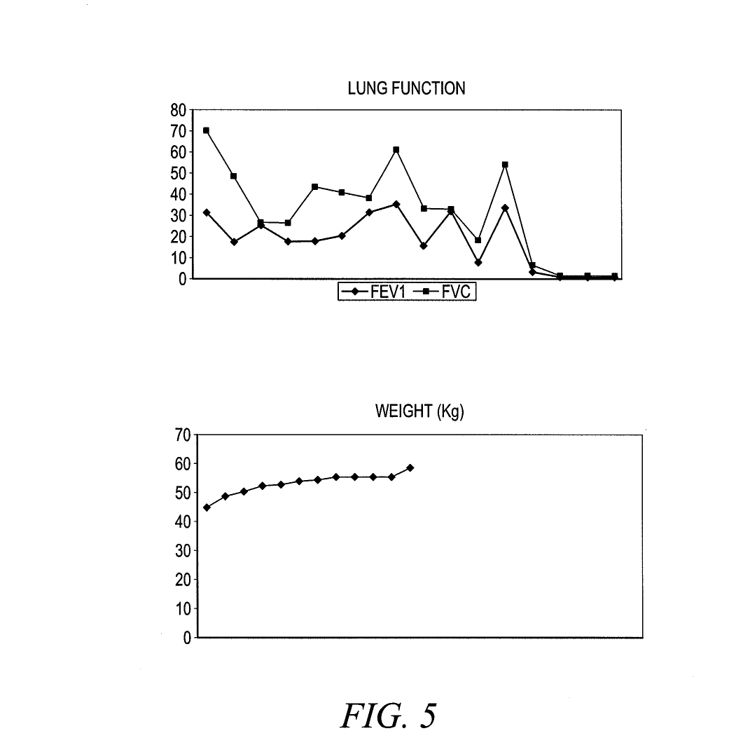

For example, if a doctor wanted to generate a severe weakness alarm, he would enter: if Appetite<3 AND (Feeble OR Tired)>7 AND Ache>7 then create alert. Alternatively, the doctor could create a sinus alert by entering: if Δ FEV1<0 AND Δ FVC<0 AND Δ Weight>0 then create alert. Even though an alert may not be generated by this rule, valuable information may still be obtained because the system 10 will analyze the parameters of the rule side by side as shown in FIG. 5.

The alert generator 42 receives the output of the rule-based analysis 26 and generates an appropriate type of alert 44 when necessary. The alert 44 can be in various forms including but not limited to email notification, pager notification, graphical output generation and other standard audio and/or visual notifications as understood by those of ordinary skill in the art. The alert 44 can be sent to either of or both of the patient 12 and the healthcare professional 18. Additionally, a record of each alert 44 that is generated by the alert generator 42 can be saved on the storage unit 20c for potential use in future analyses or on a separate alert data store 48.

After utilization of the rule-based analysis 26 and accompanying infrastructure to identify various conditions present in the patient 12, a third software can perform patient categorization and treatment plan analyses 46. These processes involve using the identified conditions present in the patient 12 to search the storage server 20 for other patients with similar conditions for purposes of disease diagnosis and medication or treatment plan development. For instance, if a patient is determined to have a particular disease, then the healthcare professional 18 can do a quick search of the existing patients who have similar symptoms. Once the search yields results, the healthcare professional 18 can quickly find the medication plans for this disease in an orderly manner and identify whether the plan can be applied for this patient 12 as well. The third software can also determine which of a selected group of patients is in the most critical condition and therefore most in need of immediate medical attention. Patient's 12 answers to questions on the heath questionnaire 16 as well as each physiological characteristic sensed can be assigned a ranking. This way, the third software can identify a patient as critical where the rank is more than a specified threshold value and medical attention can be quickly administered.

It will be understood that elements 24, 26, 40 and 42 could be implemented with appropriate software executed by server 20a, or one or more additional programmable processors all without limitation. The additional processor or processors could be proximate to server 20a, or displaced therefrom. Communication between server 20a and one or more additional processors, as noted above could be wired or wireless and/or via one or more computer networks including the Internet.

From the foregoing, it will be observed that numerous variations and modifications may be effected without departing from the spirit and scope of the invention. It is to be understood that no limitation with respect to the specific apparatus illustrated herein is intended or should be inferred. It is, of course, intended to cover by the appended claims all such modifications fall within the scope of the claims.

What is claimed is:

1. A patient monitoring system comprising:
at least one sensor of a physiological condition of an individual;
at least one transmittal interface capable of transmitting collected data from the at least one sensor to a displaced location;
a displaced data storage server;
at least one processor;
first software executable by the processor enabling analysis of validity of collected data, analysis of trends in the collected data and production of an output based on these analyses;
at least one pre-stored logical rule; and
second software executable by the processor enabling the analysis of at least some of the collected data and output of the first software based on the at least one rule to determine the presence of a condition in the individual, validation of the condition in the individual and the production of at least one output based on the outcome of the analysis and validation.

2. The system of claim 1 further comprising a user interface wherein the individual is asked at least one question about his or her physiological condition and inputs at least one answer.

3. The system of claim 2 where the at least one answer is collected and sent by the user interface to the data storage server and analyzed by the first and second software as collected data.

4. The system of claim 1 which includes a plurality of physiological sensors.

5. The system of claim 4 where members of the plurality are selected from a class that includes at least blood pressure sensors, heart rate sensors, lung function sensors, glucose sensors and PT/INR sensors.

6. The system of claim 1 wherein the data storage server includes a data storage unit with previously collected data for the individual.

7. The system of claim 6 wherein the data storage server includes a data storage unit with physiological data from a plurality of patients other than the individual.

8. The system of claim 7 wherein the data storage server includes a data storage unit with a history of all outputs previously generated by the second software.

9. The system of claim 8 wherein the second software analyzes the previously collected data for the individual, physiological data from a plurality of patients and the history of alerts in conjunction with the collected data.

10. The system of claim 7 wherein the data storage server includes a data storage unit with medication and treatment information for the plurality of other patients.

11. The system of claim 10 which includes a third software that compares the collected data for the individual to the physiological data and medication and treatment information from the plurality of other patients.

12. The system of claim 1 wherein the at least one pre-stored logical rule is created by a physician or nurse interacting with a conditional logic generator.

13. The system of claim 1 which includes a plurality of pre-stored logical rules.

14. The system of claim 13 wherein the second software segments the collected data according to each of the pre-stored logical rules to which the data pertains.

15. The system of claim 1 wherein the transmittal interface wirelessly transmits collected data to the displaced storage server.

16. The system of claim 1 where the at least one output of the second software is selected from the class including at least email notification, pager notification, graphical output generation, audio alerts and visual alerts.

17. A computer readable medium encoded with:
first software that determines whether entered data is valid and analysis of entered data to identify the presence of trends therein; and
second software that evaluates entered data according to at least one pre-stored logical rule and produces at least one output based on the outcome of the evaluation.

18. The computer readable medium of claim 15 which includes third software that assesses common trends and data points within a plurality of pre-stored data sets and sorts the data according to at least one of its characteristics.

19. A method for monitoring the physiological condition of an individual comprising:
connecting at least one physiological sensor to the individual;
transmitting the collected data from the at least one sensor to a displaced data storage server;
analyzing the collected data for validity and presence of any trends;
analyzing the collected data with at least one logical rule; and
generating at least one output based on the outcome of the analyses.

20. The method of claim 19 where the at least one logical rule is created by interacting with a conditional logic generator.

21. The system of claim 10 wherein the data storage server categorizes the patients depending on symptoms or other logical rule.

22. The system of claim 10 including software to analyze parameters of the patients and effectiveness of medication plans.
